(19) United States
(12) Patent Application Publication
(10) Pub. No.: US 2023/0210983 A1
(43) Pub. Date: Jul. 6, 2023

(54) VACCINE COMBINATION AGAINST SARS-COV-2 AND METHOD FOR PREVENTING INFECTION OF SARS-COV-2

(71) Applicant: CHANCE BIOTECHNOLOGY, INC., Taipei City (TW)

(72) Inventors: Li-Kuang CHEN, Hualien City (TW); Chuan CHIN, Taoyuan City (TW); Hui-Hua YANG, Hualien City (TW)

(21) Appl. No.: 18/149,482

(22) Filed: Jan. 3, 2023

Related U.S. Application Data
(60) Provisional application No. 63/296,670, filed on Jan. 5, 2022.

(51) Int. Cl. A61K 39/215; A61P 31/14; C07K 14/165

(52) U.S. Cl. CPC A61K 39/215; A61P 31/14; C07K 14/165; C12N 2770/20034

(57) ABSTRACT

A vaccine combination against SARS-CoV-2 includes a primer vaccine composition and a booster vaccine composition. The primer vaccine composition includes an effective amount of an Alphacoronavirus, and the primer vaccine is a live vaccine. The booster vaccine composition includes an effective amount of a Betacoronavirus, and the booster vaccine is a live vaccine.

### RELATED APPLICATIONS

This application claims priority to U.S. Provisional Application Ser. No. 63/296,670 filed Jan. 5, 2022, the disclosure of which is incorporated herein by reference in their entireties.

### BACKGROUND

### Technical Field

The present disclosure provides a vaccine combination. More particularity, the present disclosure provides a vaccine combination for preventing the infection of the SARS-CoV-2.

### Description of Related Art

COVID-19 (Coronavirus disease 2019) is an infectious disease caused by severe acute respiratory syndrome coronavirus 2 (“SARS-CoV-2” hereafter). COVID-19 is pandemic worldwide and has caused tremendous damages to the stable development of human society, and as far, more and more people died because of COVID-19.

Coronaviruses are enveloped RNA viruses that are spherical in shape and characterized by having crown-like spikes (that are, viral spike glycoproteins (VSG), “spike proteins” or “spike protein” hereafter) on the surface under an electron microscope. In taxonomy, the coronaviruses are further divided into four genera, namely Alphacoronavirus, Betacoronavirus, Gmmacoronavirus and Deltacoronavirus. There are seven human coronavirus strains, and the seven human coronavirus strains includes Coronaviruses 229E (“HCoV-229E” hereafter) and Coronaviruses NL63 (“HCoV-NL63” hereafter) belonging to Alphacoronaviruses, Coronaviruses HKU1 (“HCoV-HKU1” hereafter) and Coronaviruses OC43 (“HCoV-OC43” hereafter) belonging to Betacoronavirus, Middle East respiratory syndrome-related coronavirus (MERS-CoV), Severe acute respiratory syndrome coronavirus (SARS-CoV), and SARS-CoV-2.

Within the aforementioned seven coronavirus strains that infect humans, the clinical symptoms caused by the infections of HCoV-229E, HCoV-NL63, HCoV-HKU1 and HCoV-OC43 that cause the common cold are mild to moderate upper-respiratory tract illnesses, and may be accompanied by general feeling of being unwell, runny nose, sore throat, headache, fever, cough, etc. However, the symptoms of COVID-19 are variable, and mild-to-severe differences may appear in different patients. In detail, the common symptoms of COVID-19 are similar to those of the mild cold coronaviruses, but the serious symptoms thereof include difficulty breathing or shortness of breath, loss of speech or mobility, confusion and chest pain, and even worse, it may lead to the death of patients due to related complications.

Furthermore, the biggest challenge for controlling the spread of COVID-19 currently is that there is no vaccine which can completely prevent the infection of SARS-CoV-2, and an effective targeted therapy for COVID-19 has still yet been found. Therefore, it is urgently needed to develop effective preventing methods against this disease in order to save lives and reduce the damage provoked by this outbreak.

### SUMMARY

According to one aspect of the present disclosure, a vaccine combination against SARS-CoV-2 includes a primer vaccine composition and a booster vaccine composition. The primer vaccine composition includes an effective amount of an Alphacoronavirus, and the primer vaccine is a live vaccine. The booster vaccine composition includes an effective amount of a Betacoronavirus, and the booster vaccine is a live vaccine.

According to another aspect of the present disclosure, a method for preventing an infection of SARS-CoV-2 includes following steps. The vaccine combination against SARS-CoV-2 according to the aforementioned aspect is provided. At least one dose of the primer vaccine is administered to a subject, wherein the effective amount of the Alphacoronavirus ranges from 1×10³ pfu/ml to 1×10⁷ pfu/ml. At least one dose of the booster vaccine is administered to the subject after at least four weeks of the primer vaccine being administered, wherein the effective amount of the Betacoronavirus ranges from 1×10³ pfu/ml to 1×10⁷ pfu/ml.

### BRIEF DESCRIPTION OF THE DRAWINGS

The present disclosure can be more fully understood by reading the following detailed description of the embodiment, with reference made to the accompanying drawings as follows:

FIG. 1 is a flow chart of a method for preventing an infection of SARS-CoV-2 according to another aspect of the present disclosure.

FIG. 2 shows the seroconversion rate of the voluntary subjects after accepting the vaccine combination against SARS-CoV-2 of the present disclosure for eight weeks.

FIG. 3 shows the diagram of the flow cytometry analysis of cells in the peripheral blood of Subject 1 after accepting the vaccine combination against SARS-CoV-2 of the present disclosure for four weeks.

FIG. 4 shows the diagram of the flow cytometry analysis of the antigen distribution of T cells of Subject 1 after accepting the vaccine combination against SARS-CoV-2 of the present disclosure for four weeks.

### DETAILED DESCRIPTION

The present disclosure will be further exemplified by the following specific embodiments to facilitate utilizing and practicing the present disclosure completely by the people skilled in the art without over-interpreting and over-experimenting. However, these practical details are used to describe how to implement the materials and methods of the present disclosure and are not necessary.

<Vaccine Combination Against SARS-CoV-2 of the Present Disclosure>

According to one aspect of the present disclosure, a vaccine combination against SARS-CoV-2 includes a primer vaccine composition and a booster vaccine composition.

The primer vaccine composition includes an effective amount of an Alphacoronavirus, and the primer vaccine composition is a live vaccine. In detail, the effective amount of the Alphacoronavirus in the primer vaccine composition can range from 1×10³ pfu/ml to 1×10⁷ pfu/ml, and the Alphacoronavirus can be HCoV-229E, HCoV-NL63, or a combination thereof. Further, the primer vaccine composition can be an oral vaccine, but the present disclosure is not limited thereto.

The booster vaccine composition includes an effective amount of a Betacoronavirus, and the booster vaccine composition is a live vaccine. In detail, the effective amount of the Betacoronavirus in the booster vaccine composition can range from 1×10³ pfu/ml to 1×10⁷ pfu/ml, and the Betacoronavirus can be HCoV-HKU1, HCoV-OC43, or a combination thereof. Further, the booster vaccine composition can be a nasal spray vaccine, but the present disclosure is not limited thereto.

In particular, the spike protein of SARS-CoV-2 is mainly used as the target antigen in the development of the vaccine against SARS-CoV-2 currently. However, according to the clinical results of current vaccinations, after the user accepted the vaccine using the SARS-CoV-2 spike protein or the nucleotide encoding the spike protein as the antigen, there is still a high probability of being infected by the mutant strain of SARS-CoV-2 and resulting in the breakthrough infection. Thus, it is shown that the active immune response induced solely by the antigen of the spike protein of SARS-CoV-2 or the nucleotide encoding the spike protein cannot be effective against the infection of different SARS-CoV-2 variant strains. Therefore, the vaccine combination against SARS-CoV-2 of the present disclosure uses the whole virus of milder and infectiously active human mild cold coronaviruses as the antigen, so that the human mild cold coronaviruses can infect the human body naturally, and the active immune response in the human body can be induced to produce anti-coronavirus antibodies so as to obtain more extensive and comprehensive protection.

Please refer to Table 1, which shows the protein sequence identity of the spike proteins between SARS-CoV-2 and the human mild cold coronaviruses HCoV-229E, HCoV-NL63, HCoV-HKU1, and HCoV-OC43.

TABLE 1

| Human mild cold coronaviruses | | Sequence identity of spike protein |
|---|---|---|
| Virus | Genus | compared with SARS-CoV-2 (%) |
| HCoV-229E | Alphacoronavirus | 27.78 |
| HCoV-NL63 | Alphacoronavirus | 31.27 |
| HCoV-OC43 | Betacoronavirus | 37.65 |
| HCoV-HKU1 | Betacoronavirus | 36.66 |

As shown in Table 1, HCoV-229E, HCoV-NL63, HCoV-HKU1 and HCoV-OC43 share at least 27.78% identity of the sequence of the spike proteins to the SARS-CoV-2, and the sequence identity of the spike proteins between HCoV-OC43 and SARS-CoV-2 can reach 37.65%. Further, in the proteins of coronaviruses, RNA-polymerase within the early transcribed replication transcription complex (RTC) thereof is the largest region of high sequence conservation across the human mild cold coronaviruses and SARS-CoV-2 clades, and other structural proteins and non-structural proteins of the human mild cold coronaviruses are also highly conserved with those of the SARS-CoV-2. Thus, the infection of the human mild cold coronaviruses will provide the immunity against SARS-CoV-2 due to the interactions with the spike protein and more than twenty other candidate proteins thereof.

Furthermore, as shown in the clinical data, the human mild cold coronaviruses will naturally infect the subject, and the RTC regions, expressed in the first stage of the viral life cycle, are highly conserved among human coronaviruses and are preferentially targeted by T cells so as to induce the active immune response in the human body. The T cells generated from the aforementioned active immune response can cross-recognize the RTC epitopes of SARS-CoV-2 and human mild cold coronaviruses. Thus, it is shown that the infection of the human mild cold coronaviruses can lead to the generation of the T cells with the cross-recognition ability for SARS-CoV-2 in vivo and in advance. In other words, T cells generated from the infection of the whole virus of the human mild cold coronaviruses can contribute to the rapid clearance of SARS-CoV-2 and other coronavirus infections.

Therefore, under the strategy of the heterologous prime/boost vaccination, the primer vaccine composition and the booster vaccine composition of the vaccine combination against SARS-CoV-2 of the present disclosure respectively include the coronaviruses belonging to different genera. It is favorable for more completely inducing the active immune response of the human body against different types of coronaviruses, and the aim of resisting the infection of SARS-CoV-2 can be achieved. Preferably, the Alphacoronavirus can be HCoV-229E, the Betacoronavirus can be HCoV-OC43, the effective amount of the Alphacoronavirus can be 1×10⁵ pfu/ml, the effective amount of the Betacoronavirus can be 1×10⁵ pfu/ml, and the present disclosure is not limited thereto.

<Method for Preventing an Infection of SARS-CoV-2 of the Present Disclosure>

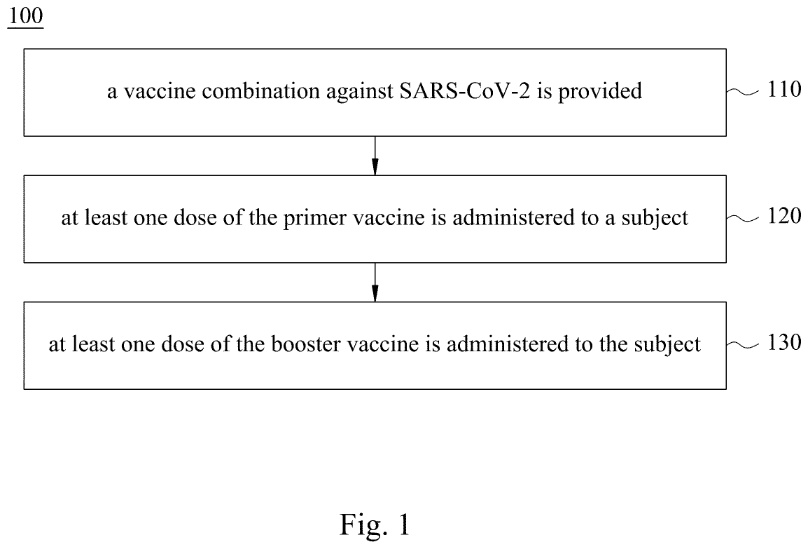

Reference is made to FIG. 1, which is a flow chart of a method 100 for preventing an infection of SARS-CoV-2 according to another aspect of the present disclosure. The method 100 for preventing the infection of SARS-CoV-2 includes Step 110, Step 120 and Step 130.

In Step 110, a vaccine combination against SARS-CoV-2 is provided. In detail, the vaccine combination against SARS-CoV-2 is the vaccine combination according to the aforementioned aspect, and the vaccine combination includes the primer vaccine composition and the booster vaccine composition as foregoing described. Thus, the details thereof will not be described again herewith.

In Step 120, at least one dose of the primer vaccine is administered to a subject, wherein the effective amount of the Alphacoronavirus ranges from 1×10³ pfu/ml to 1×10⁷ pfu/ml.

In Step 130, at least one dose of the booster vaccine is administered to the subject after at least four weeks of the primer vaccine being administered, wherein the effective amount of the Betacoronavirus ranges from 1×10³ pfu/ml to 1×10⁷ pfu/ml.

In detail, the primer vaccine composition of the present disclosure uses the whole and alive virus of the Alphacoronavirus as the antigen, so that the Alphacoronavirus will infect the subject naturally so as to induce the active immune response in the human body. After at least four weeks after the vaccination of the primer vaccine, the booster vaccine including the whole and alive virus of the Betacoronavirus is applied to the subject so as to facilitate the Betacoronavirus to infecting the subject naturally. Then, the subject can obtain the immunity against the coronaviruses including SARS-CoV-2, and the immunity can last for a long time.

Therefore, by applying the primer vaccine composition and the booster vaccine composition of the vaccine combination against SARS-CoV-2 of the present disclosure, wherein the primer vaccine composition and the booster vaccine composition respectively include the coronaviruses belonging to different genera, and the primer vaccine composition and the booster vaccine composition are administered sequentially at least four weeks apart, the method 100 for preventing the infection of SARS-CoV-2 of the present disclosure can more completely induce the active immune response of the human body against different types of coronaviruses. Thus, it has excellent potential to prevent the infection of SARS-CoV-2.

<Analysis of the Anti-Coronavirus Effects of the Vaccine Combination Against SARS-CoV-2 of the Present Disclosure>

In the present experiment, the vaccine combination against SARS-CoV-2 includes the primer vaccine composition and the booster vaccine composition, wherein the primer vaccine composition includes the Alphacoronavirus HCoV-229E, and the booster vaccine composition includes the Betacoronavirus HCoV-OC43. Further, the effective amount of the Alphacoronavirus of the primer vaccine composition is 1×10⁵ pfu/ml, and the effective amount of the Betacoronavirus of the booster vaccine composition is 1×10⁵ pfu/ml. Furthermore, the primer vaccine composition is applied by an oral route, and the booster vaccine composition is applied by a nasal spray route.

I. Analysis of the Titrations of Antibodies

In the present experiment, 237 voluntary subjects are vaccinated with the primer vaccine of the vaccine combination against SARS-CoV-2 of the present disclosure, and after four weeks of the administration of the primer vaccine, the booster vaccine of the vaccine combination against SARS-CoV-2 of the present disclosure is applied to the 237 voluntary subjects. After eight weeks of the vaccination, the titrations of antibodies against HCoV-229E and HCoV-OC43 in the blood of the voluntary subjects are measured.

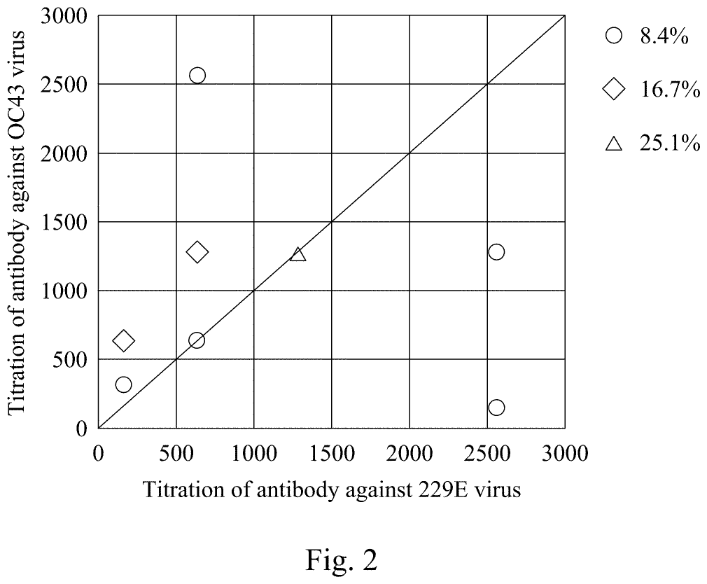

Reference is made to FIG. 2, which shows the seroconversion rate of the voluntary subjects after accepting the vaccine combination against SARS-CoV-2 of the present disclosure for eight weeks. As shown in FIG. 2, after eight weeks of the vaccination of the vaccine combination against SARS-CoV-2 of the present disclosure, the titrations of antibodies against HCoV-229E (“229E virus” in FIG. 2) and HCoV-OC43 (“OC43 virus” in FIG. 2) are excellent, and there is 25.1% of all the 237 voluntary subjects having the titrations of antibodies of both HCoV-229E and HCoV-OC43 larger than 1000 simultaneously. Accordingly, it is shown that the vaccine combination against SARS-CoV-2 and the method for preventing the infection of SARS-CoV-2 of the present disclosure can effectively induce an active immune response in the human body against different types of coronaviruses, and the produced amount of the antibodies is also high.

II. Analysis of the Side Effects

In the present experiment, the situation of side effects in the aforementioned 237 voluntary subjects after eight weeks of the vaccination of the vaccine combination against SARS-CoV-2 of the present disclosure is observed, and the results thereof are shown in Table 2.

TABLE 2

| | Number of cases/ |
|---|---|
| Side effects | Proportion of all subjects |
| Mild upper respiratory tract infection | 5/2.1% |
| Gastrointestinal upset | 3/1.3% |
| (Diarrhea ≤2 times) | |
| Allergic skin rash | 3/1.3% |
| Fever | 0/0% |
| Pneumonia | 0/0% |
| CNS abnormality | 0/0% |

As shown in Table 2, after accepting the vaccine combination against SARS-CoV-2 of the present disclosure, less than 3% of the voluntary subjects had mild upper respiratory tract infection symptoms, and 1.3% of the voluntary subjects had the side effects of the gastrointestinal upset and the allergic skin rash. Accordingly, it is shown that the vaccine combination against SARS-CoV-2 of the present disclosure will not induce serious side effects like that of the current clinical vaccine against SARS-CoV-2, and has excellent biological safety.

III. Analysis of T Cells

In the present experiment, 484 voluntary subjects are vaccinated with the primer vaccine of the vaccine combination against SARS-CoV-2 of the present disclosure, and after four weeks of the administration of the primer vaccine, the booster vaccine of the vaccine combination against SARS-CoV-2 of the present disclosure is applied to the 484 voluntary subjects. After eight weeks of the administration of the booster vaccine composition, the peripheral blood of one of the voluntary subjects (“Subject 1” hereafter) is collected, and expression of cells in the peripheral blood and the surface antigen performance of T cells thereof are analyzed.

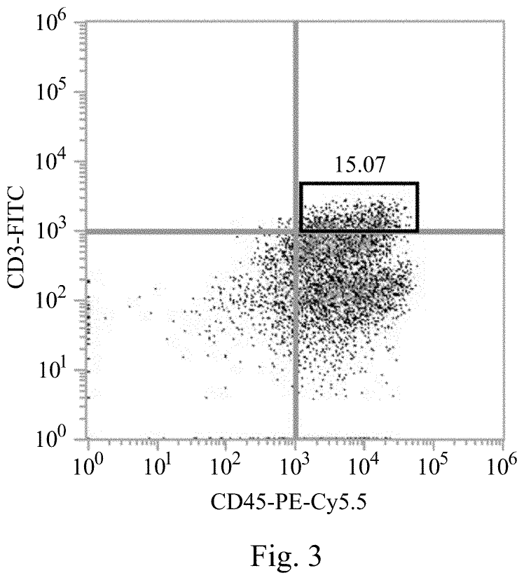

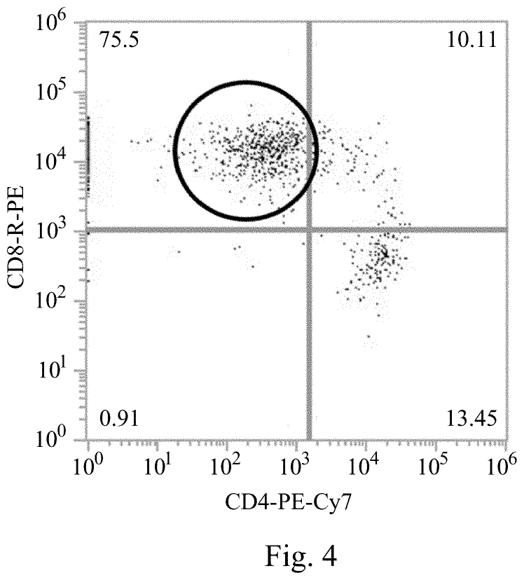

Reference is made to FIG. 3 and FIG. 4, wherein FIG. 3 shows the diagram of the flow cytometry analysis of cells in the peripheral blood of Subject 1 after accepting the vaccine combination against SARS-CoV-2 of the present disclosure for four weeks, and FIG. 4 shows the diagram of the flow cytometry analysis of the antigen distribution of T cells of Subject 1 after accepting the vaccine combination against SARS-CoV-2 of the present disclosure for four weeks.

As shown in FIG. 3, after analyzing the cells in the peripheral blood of Subject 1, activated T cells expressing CD3 (CD3-FITC) and CD45 (CD45-PE-Cy5.5) (the cells marked by the box in FIG. 3) are about 15.07% of the total cells. Further, as shown in FIG. 4, after further analyzing the T cells of 15.07% of the total cells, the T cells expressing CD8 (CD8-R-PE) but not CD4 (CD4-PE-Cy7) in the peripheral blood of Subject 1 (the cells marked by the circle in FIG. 4) are about 75.5% of the total T cells. Thus, it is shown that the T cells in the peripheral blood of Subject 1 can recognize the epitopes of the coronavirus and are specific to the coronavirus, so that the infection of the coronavirus can be effectively prevented.

Further, the saliva of 25 of the voluntary subjects is collected, wherein the 25 voluntary subjects had no history of SARS-CoV-2 infection, nor had been vaccinated with commercial vaccinations of COVID-19. The commercial kit, COVID-19 Antibody Rapid Test (BORDER ZERO©) (TOA Industry Co., Ltd. Japan), is used for analyzing the expression of IgA in the saliva of the 25 voluntary subjects.

Reference is made to Table 3, which shows the expression of IgA in the saliva of the 25 voluntary subjects after being vaccinated for four weeks.

TABLE 3

| | | Number of cases/ |
|---|---|---|
| Signal intensity of IgA in saliva | | Proportion of all subjects |
| Positive | Strong | 3/12% |
| | Medium | 16/64% |
| | Weak | 5/20% |
| Negative | Unable to detect | 1/4% |

As shown in Table 3, up to 96% of the 25 voluntary subjects have expressed IgA in the saliva, and it is shown that the vaccine combination against SARS-CoV-2 and the method for preventing the infection of SARS-CoV-2 of the present disclosure can effectively induce the mucosal immunity against SARS-CoV-2 in the voluntary subjects, and there are related clinical application potential thereof.

As shown above, the vaccine combination against SARS-CoV-2 and the method for preventing the infection of SARS-CoV-2 of the present disclosure use the whole virus of milder and infectiously active human mild cold coronaviruses as the antigen so as to induce the active immune response in the human body. It has a superior ability against SARS-CoV-2 infection compared to the current vaccines using the spike protein or the nucleotides encoding spike proteins as antigens. Further, the primer vaccine composition and the booster vaccine composition of the vaccine combination against SARS-CoV-2 of the present disclosure are applied sequentially, and the primer vaccine composition and the booster vaccine composition respectively include the coronaviruses belonging to different genera. Thus, it is favorable for more completely inducing the active immune response of the human body against different types of coronaviruses, so that the aim of resisting the infection of SARS-CoV-2 can be achieved.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure covers modifications and variations of this disclosure provided they fall within the scope of the following claims.

What is claimed is:

1. A vaccine combination against SARS-CoV-2, comprising:
a primer vaccine composition, comprising:
an effective amount of an Alphacoronavirus, wherein the primer vaccine is a live vaccine; and
a booster vaccine composition, comprising:
an effective amount of a Betacoronavirus, wherein the booster vaccine is a live vaccine.

2. The vaccine combination against SARS-CoV-2 of claim 1, wherein the primer vaccine is an oral vaccine.

3. The vaccine combination against SARS-CoV-2 of claim 1, wherein the booster vaccine is a nasal spray vaccine.

4. The vaccine combination against SARS-CoV-2 of claim 1, wherein the effective amount of the Alphacoronavirus ranges from 1×10³ pfu/ml to 1×10⁷ pfu/ml.

5. The vaccine combination against SARS-CoV-2 of claim 1, wherein the effective amount of the Betacoronavirus ranges from 1×10³ pfu/ml to 1×10⁷ pfu/ml.

6. The vaccine combination against SARS-CoV-2 of claim 1, wherein the Alphacoronavirus is HCoV-229E, HCoV-NL63, or a combination thereof.

7. The vaccine combination against SARS-CoV-2 of claim 6, wherein the Alphacoronavirus is HCoV-229E.

8. The vaccine combination against SARS-CoV-2 of claim 1, wherein the Betacoronavirus is HCoV-HKU1, HCoV-OC43, or a combination thereof.

9. The vaccine combination against SARS-CoV-2 of claim 8, wherein the Betacoronavirus is HCoV-OC43.

10. A method for preventing an infection of SARS-CoV-2, comprising:
providing the vaccine combination against SARS-CoV-2 of claim 1;
administering at least one dose of the primer vaccine to a subject, wherein the effective amount of the Alphacoronavirus ranges from 1×10³ pfu/ml to 1×10⁷ pfu/ml; and
administering at least one dose of the booster vaccine to the subject after at least four weeks of the primer vaccine being administered, wherein the effective amount of the Betacoronavirus ranges from 1×10³ pfu/ml to 1×10⁷ pfu/ml.

11. The method of claim 10, wherein the primer vaccine is an oral vaccine.

12. The method of claim 10, wherein the booster vaccine is a nasal spray vaccine.

13. The method of claim 10, wherein the Alphacoronavirus is HCoV-229E, HCoV-NL63, or a combination thereof.

14. The method of claim 13, wherein the Alphacoronavirus is HCoV-229E.

15. The method of claim 10, wherein the Betacoronavirus is HCoV-HKU1, HCoV-OC43, or a combination thereof.

16. The method of claim 15, wherein the Betacoronavirus is HCoV-OC43.
